(19) United States
(12) Patent Application Publication
(10) Pub. No.: US 2010/0137972 A1
(43) Pub. Date: Jun. 3, 2010

(54) SINTERED MOULDED BODY

(76) Inventors: Meinhard Kuntz, Esslingen (DE); Ana Herrán Fuertes, Stuttgart (DE); Kilian Friederich, Plochingen (DE); Norbert Schneider, Schorndorf (DE)

Correspondence Address: FULBRIGHT & JAWORSKI, LLP, 666 FIFTH AVE, NEW YORK, NY, 10103-3198, US

(21) Appl. No.: 12/597,557

(22) PCT Filed: Apr. 25, 2008
(86) PCT No.: PCT/EP08/55059

§ 371 Date: Oct. 26, 2009

(30) Foreign Application Priority Data
Apr. 27, 2007 (DE) 10 2007 020 471.1

(51) Int. Cl. C04B 35/106; C04B 35/80; B32B 5/02; A61F 2/82; A61F 2/30

(52) U.S. Cl. 623/1.15; 501/104; 501/95.1; 442/59; 623/18.11

(57) ABSTRACT

A sintered molded body consisting of a material that contains aluminum oxide with chromium doping, zirconium oxide with Y-stabilization and strontium aluminates with variable Cr-doping, which is particularly suitable for medical applications.

The invention relates to a sintered moulding.

Sintered mouldings offer a wide range of possible applications. Their composition can be adapted to their intended use by the targeted addition of specific elements and/or compounds thereof. Aluminium oxide and zirconium oxide, for example, are ceramic materials which, individually or in combination with one another, can be processed into sintered mouldings such as cutting tools, catalyst supports or prostheses.

The object of the invention is to provide a sintered moulding made of a ceramic material which combines optimum properties such as hardness, elasticity and thermal conductivity and is particularly suitable for medical technology applications.

Surprisingly, it has been shown that a sintered moulding of the following composition is particularly suitable for use in the field of medical technology, for example for use as an orthosis or endoprosthesis, such as hip and knee joint implants.

| | | Volume |
|---|---|---|
| Material composition | Formula | percentage |
| Aluminium oxide with chromium doping | Al₂O₃:Cr | 70%-90% |
| Zirconium oxide with Y stabilisation | ZrO₂:Y | 12%-22% |
| Strontium aluminate (with variable Cr | SrAl₁₂₋ₓCrₓO₁₉ | 1%-5% |
| doping) | | |

The dominant structural component of a sintered moulding of this type is aluminium oxide. The property-determining features, such as hardness, modulus of elasticity and thermal conductivity, are therefore very close to the properties of pure aluminium oxide. The components zirconium oxide and strontium aluminate are embedded in the aluminium oxide matrix. The strontium aluminate forms characteristic plate-like crystallites, platelets, which make a significant contribution to the increase in strength.

The components zirconium oxide and strontium aluminate contribute to the increase in fracture toughness, which is about 60% higher than is the case with pure aluminium oxide. These reinforcing components result in an increase in strength by a factor of almost 2, and at the same time the damage tolerance, i.e. the property of the sintered moulding to retain high residual strength even with possible damage, also increases.

When the sintered compact according to the invention is under high mechanical stress, mechanisms are surprisingly activated which, for example, inhibit or stop crack propagation. The most important mechanism here is the stress-induced conversion of the zirconium oxide from the tetragonal to the monoclinic phase. The volume expansion of the zirconium oxide associated with the conversion causes the formation of local compressive stresses, which counteracts the external tensile load and thus prevents crack growth.

Surprisingly, the crack path is deflected by the embedded platelets, and so additional energy is absorbed during crack propagation.

It may be regarded as a special feature of the sintered moulding according to the invention that the two mechanisms mutually reinforce one another so that the effective increase in fracture toughness is even greater than would be expected from the simple addition of the individual mechanisms.

The production of sintered mouldings takes place by conventional ceramics technology. The essential process steps are:
a) Adding the powder mixture to water in the specified composition, using liquefiers to avoid sedimentation.
b) Homogenising in a high-speed mixer.
c) Grinding in an attrition mill, thus increasing the specific surface area of the powder mixture (=comminution).
d) Adding organic binders.
e) Spray-drying, resulting in free-flowing granules with defined properties.
f) Moistening the granules with water.
g) Pressing axially or isostatically.
h) Green machining, largely forming the final contours taking into account the shrinkage on sintering.
i) Pre-firing, during which shrinkage to approx. 98% of the theoretical density occurs. Any remaining residual pores are closed to the outside.
j) Hot isostatic pressing at high temperature and under high gas pressure, resulting in almost complete final compression.
k) So-called white firing, resulting in equalisation of the imbalance of the oxygen ions in the ceramic produced during hot isostatic pressing.
l) Hard machining by grinding and polishing.
m) Annealing.

The properties of the sintered moulding can be further reinforced by means of inclusions. Thus, it is possible to mix whiskers and/or fibres into the material before shaping a sintered compact, or to incorporate net-like structures or woven fabrics into the material in the green state. The whiskers, fibres or nets or woven fabrics must be made of a material which does not interact with the ceramic material in a way that would lead to an impairment of its properties. Furthermore, the material must not become modified during sintering in a way that would damage the material.

The sintered mouldings according to the invention surprisingly combine the best properties of sintered mouldings of pure aluminium oxide and zirconium oxide for implant applications: hardness, ageing resistance, wetting behaviour with respect to water and high thermal conductivity are properties known from sintered mouldings of aluminium oxide, and high strength and high fracture toughness, i.e. damage tolerance, are properties known from sintered mouldings of zirconium oxide.

1-7. (canceled)

8. A sintered molding comprising 70 to 90 parts by volume aluminium oxide with chromium doping (Al₂O₃:Cr), 12 to 22 parts by volume zirconium oxide with Y stabilisation (ZrO₂:Y) and 1 to 5 parts by volume strontium aluminate having the formula SrAl₁₂₋ₓCrₓO₁₉ with variable Cr doping.

9. A sintered molding according to claim 8, wherein the components zirconium oxide and strontium aluminate are embedded in the aluminium oxide matrix.

10. A sintered molding according to claim 8, wherein the strontium aluminate is present in the form of plate-like crystallites, platelets.

11. A sintered molding according to claim 8, wherein the material is additionally interspersed with whiskers, fibers, net-like structures or woven fabrics made of suitable materials.

12. A medical device comprising the sintered molding according to claim 8.

13. An orthosis and endoprosthesis comprising the sintered molding of claim 8.

14. A hip or knee joint implant comprising the sintered molding of claim 8.

15. A sintered molding according to claim 9, wherein the strontium aluminate is present in the form of plate-like crystallites, platelets.

16. A sintered molding according to claim 9, wherein the material is additionally interspersed with whiskers, fibers, net-like structures or woven fabrics made of suitable materials.

17. A medical device comprising the sintered molding according to claim 9.

18. An orthosis and endoprosthesis comprising the sintered molding of claim 9.

19. A hip or knee joint implant comprising the sintered molding of claim 9.

20. A sintered molding according to claim 10, wherein the material is additionally interspersed with whiskers, fibers, net-like structures or woven fabrics made of suitable materials.

21. A medical device comprising the sintered molding according to claim 10.

22. An orthosis and endoprosthesis comprising the sintered molding of claim 9.

23. A hip or knee joint implant comprising the sintered molding of claim 9.

24. A sintered molding according to claim 10, wherein the material is additionally interspersed with whiskers, fibers, net-like structures or woven fabrics made of suitable materials.

25. A medical device comprising the sintered molding according to claim 10.

26. An orthosis and endoprosthesis comprising the sintered molding of claim 10.

27. A hip or knee joint implant comprising the sintered molding of claim 10.
